(19) United States
(12) Patent Application Publication
(10) Pub. No.: US 2011/0105523 A1
(43) Pub. Date: May 5, 2011

(54) BIFEPRUNOX DERIVATIVES

(76) Inventors: Herman H. Van Stuivenberg, Weesp (NL); Gerrit A. Barf, Weesp (NL)

(21) Appl. No.: 12/812,788

(22) PCT Filed: Jan. 14, 2009
(86) PCT No.: PCT/EP09/50336

§ 371 Date: Jan. 14, 2011

Related U.S. Application Data
(60) Provisional application No. 61/021,090, filed on Jan. 15, 2008.

(30) Foreign Application Priority Data
Jan. 15, 2008 (EP) 08150282.5

(51) Int. Cl. C07D 413/10; A61K 31/496; A61P 25/00; A61P 25/16; A61P 9/12; A61P 5/00; A61P 25/18; A61P 35/04; A61P 17/00

(52) U.S. Cl. 514/254.02; 544/368

(57) ABSTRACT

The present invention relates to bifeprunox derivatives of formula (I) wherein R1 is one substituent chosen from 3-OH, 4-OH, 3-OSO3H and 4-OSO3H; R2 is H; or an N-oxide or a pharmaceutically acceptable salt, or a solvate or hydrate of any of the foregoing. The compounds of the invention may be used in the treatment or alleviation of dopamine D₂ receptor mediated diseases or conditions.

### FIELD OF THE INVENTION

This invention relates to new derivatives of bifeprunox, a pharmaceutical composition containing said compounds, as well as the use of said compounds for the preparation of a medicament for treating, alleviating or preventing diseases and conditions mediated by dopamine D₂ and 5-HT_{1A} receptors.

### BACKGROUND OF THE INVENTION

Bifeprunox (7-[4-([1,1′-biphenyl]-3-ylmethyl1)-1-piperazinyl]-2(3H)-benzoxazolone monomethanesulphonate, see e.g. Bioorg. & Med. Chem. Lett. 11 (2001), 2345-2349, compound 5b; WO 97/36893, Table A1, compound A2) is an atypical antipsychotic with a unique pharmacologic profile. It is a highly potent partial dopamine D₂ agonist with moderately potent partial 5-HT_{1A} agonist activity.

### DESCRIPTION OF THE INVENTION

It has now been found that bifeprunox derivatives of the formula (I)

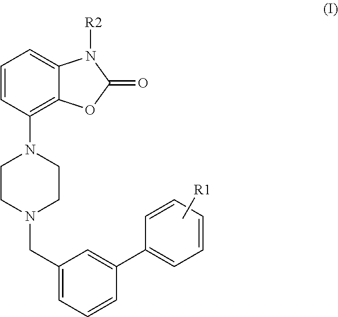

wherein

R1 is one substituent selected from 3-OH, 4-OH, 3-OSO₃H and 4-OSO₃H;

R2 is H;

or an N-oxide or a pharmaceutically acceptable salt, a solvate or hydrate thereof are potent ligands for the dopamine D₂ receptor, exhibiting strong partial dopamine D₂ agonistic effects with a percentage agonism significantly higher than that of bifeprunox.

The compounds of the invention are useful for treating, alleviating and preventing dopamine D₂ receptor mediated diseases and conditions, where D₂ receptor agonistic effects are needed. In particular, compounds of the present invention may be used to treat, alleviate or prevent CNS related diseases where D₂ receptor agonistic effects are needed, such as—but not limited to—Parkinson's disease and Restless Leg Syndrome (RLS; also known as Ekbom's syndrome), and in particular Parkinson's disease.

Some compounds display in particular non-CNS D₂ receptor agonistic effects and may be useful in: (1) the treatment of hypertension, including but not limited to, its use both orally and intravenously to increase cardiac outflow after cardiac surgery, in heart failure, in cardiogenic shock and cirrhotic ascites, to improve renal function and in the prevention of renal failure (Semeraro et al, Clin Exp Hypertens. 1997 January-February; 19(1-2):201-15; Luchsinger et al, Am J Ther. 1998 March; 5(2):81-8; O'Connell & Aherne, Clin Exp Hypertens. 2000 April; 22(3):217-49; Doggrell, Expert Opin Investig Drugs. 2002 May; 11(5):631-44); (2) the treatment of acromegaly resulting from the hypersecretion of growth hormone caused by pituitary adenomas (Diez et al, Expert Opin Pharmacother. 2000 July; 1(5):991-1006; Cap et al, Cas Lek Cesk. 2005; 144 Suppl 3:33-4, 36-7. [In Czech.]); (3) the treatment of hyperprolactinaemia arising from all causes; hyperprolactinaemia produces the clinical symptoms of hypogonadism, which manifests itself as fertility disturbances (for instance in the menstruation cycle), oligomenorrhea or amenorrhea in women, and libido loss, impotence, and fertility disturbances in men, as well as bone density disturbances (osteopenia, osteoporosis) and galactorrhea (Webster, Baillieres Best Pract Res Clin Endocrinol Metab. 1999 October; 13(3):395-408; Kaluzny et al, Postepy Hig Med Dosw (Online). 2005; 59:20-7. [In Polish]); further, hyperprolactinaemia may be related to breast cancer; (4) to reduce the size of and in the management of pituitary adenomas, particularly prolactin secreting adenomas, including but not limited to, micro- and macroprolactinomas and non-secreting prolactinomas (Webster, Baillieres Best Pract Res Clin Endocrinol Metab. 1999 October; 13(3):395-408; Di Sarno et al, Clin Endocrinol (Oxf). 2000 July; 53(1):53-60; Bolko et al, Pol Arch Med. Wewn. 2003 May; 109(5):489-95; Kaluzny et al, Postepy Hig Med Dosw (Online). 2005; 59:20-7. [In Polish]); (5) the treatment of hyperprolactinaemia caused by administration of typical neuroleptic drugs, atypical antipsychotics and other dopamine D2 receptor antagonists (Cohen & Biederman, J Child Adolesc Psychopharmacol. 2001 Winter; 11(4):435-40); (6) the treatment of ovarian hyperstimulation syndrome (OHSS), which results from ovarian over-expression of vascular endothelial growth factor (VEGF) and its receptor 2 (VEGFR2), and in particular, preventing and treatment of haemoconcentration and ascites in women with ovarian hyperstimulation undergoing assisted reproduction in fertility treatment (Alvarez et al, Hum Reprod. 2007 Oct. 4; [Epub ahead of print] and J Clin Endocrinol Metab. 2007 August; 92(8):2931-7); (7) the prevention of cell proliferation (tumour growth) in small-cell lung carcinomas (Senogles et al, Anticancer Drugs. 2007 August; 18(7):801-7); (8) the prevention and treatment of multi-drug resistance in cancer chemotherapy (Shiraki et al, Jpn J Cancer Res. 2002 February; 93(2):209-15); (9) in dermatology, particularly to accelerate barrier repair and inhibit the epidermal hyperplasia induced by barrier disruption (Fuziwara et al, J Invest Dermatol. 2005 October; 125(4):783-9).

In a preferred embodiment of the invention, the compounds have formula (I) wherein R1 is 3-OH or 4-OH and R2 is H.

In another embodiment of the invention, the compounds have formula (I) wherein R1 is 3-OSO₃H or 4-OSO₃H and R2 is H. These compounds are particularly useful in non-CNS indications.

The compounds of the invention may suitably be prepared by methods available in the art, as illustrated the scheme of FIG. 1 and in the experimental section of this description.

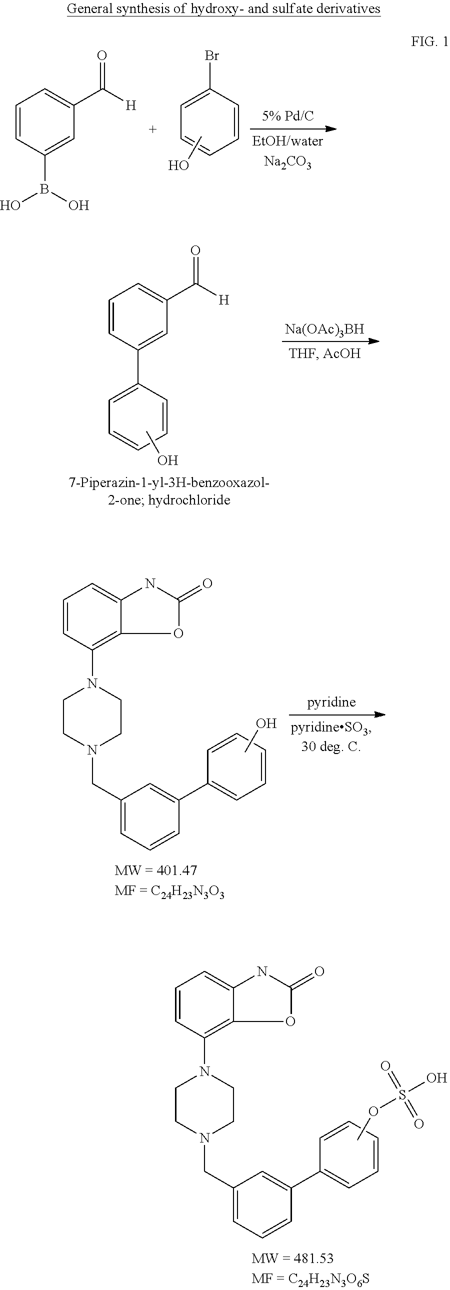

N-oxides of the compounds of the formula (I) may be prepared by the methods described for the preparation of the N-oxide of bifeprunox (WO 2007/023141).

The compounds of the invention may exist as polymorphs and as such are intended to be included in the present invention. In addition, compounds may form solvates with water (i.e., hydrates) or common organic solvents, and such solvates are also intended to be encompassed within the scope of this invention.

Isotopically-labeled compound of formula (I) or pharmaceutically acceptable salts thereof, including compounds of formula (I) isotopically-labeled to be detectable by PET or SPECT, also fall within the scope of the invention. The same applies to compounds of formula (I) labeled with [¹³C]—, [¹⁴C]—, [³H]— or other isotopically enriched atoms, suitable for receptor binding or metabolism studies.

The term “pharmaceutically acceptable salt” refers to those salts that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids.

The compounds of the invention may be administered enterally or parenterally. The exact dose and regimen of these compounds and compositions thereof will be dependent on the biological activity of the compound per se, the age, weight and sex of the patient, the needs of the individual subject to whom the medicament is administered, the degree of affliction or need and the judgment of the medical practitioner. In general, parenteral administration requires lower dosages than other methods of administration which are more dependent upon adsorption. However, the dosages for humans are preferably 0.001-10 mg per kg body weight, more preferably 0.01-1 mg per kg body weight. In general, enteral and parenteral dosages will be in the range of 0.1 to 1,000 mg per day of total active ingredients. The medicament manufactured with the compounds of this invention may also be used as adjuvant in therapy. In such a case, the medicament or is administered in a combination treatment with other compounds useful in treating such disease states. Also pharmaceutical combination preparations comprising at least one compound of the present invention and at least one other pharmacologically active substance are considered in this respect.

Mixed with pharmaceutically suitable auxiliaries, e.g. as described in the standard reference “Remington, The Science and Practice of Pharmacy” (21^{st} edition, Lippincott Williams & Wilkins, 2005, see especially Part 5: Pharmaceutical Manufacturing) the compounds may be compressed into solid dosage units, such as pills or tablets, or be processed into capsules or suppositories. By means of pharmaceutically suitable liquids the compounds can also be applied in the form of a solution, suspension or emulsion.

For making dosage units, e.g. tablets, the use of conventional additives such as fillers, colorants, polymeric binders and the like, is contemplated. In general, any pharmaceutically suitable additive which does not interfere with the function of the active compounds can be used.

Suitable carriers with which the compounds of the invention can be administered include for instance lactose, starch, cellulose derivatives and the like, or mixtures thereof, used in suitable amounts. Compositions for intravenous administration may for example be solutions of the compounds of the invention in sterile isotonic aqueous buffer. Where necessary, the intravenous compositions may include for instance solubilizing agents, stabilizing agents and/or a local anesthetic to ease the pain at the site of the injection.

Pharmaceutical compositions of the invention may be formulated for any route of administration and comprise at least one compound of the present invention and pharmaceutically acceptable salts thereof, with any pharmaceutically suitable ingredient, excipient, carrier, adjuvant or vehicle.

By “pharmaceutically suitable” it is meant that the carrier, diluent or excipient must be compatible with the other ingredients of the formulation and not deleterious to the recipient thereof.

In an embodiment of the invention, a pharmaceutical pack or kit is provided comprising one or more containers filled with one or more pharmaceutical compositions of the invention. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described in this document.

The following examples are only intended to further illustrate the invention in more detail.

### EXAMPLES

### §1. Abbreviations

Ac acetyl
DCM dichloromethane
DEM diethoxymethane
DMSO dimethylsulfoxide
EtOH ethanol
EtOAc ethyl acetate
g gram(s)
h hour(s)
min minute(s)
THF tetrahydrofurane

### Abbreviations in ¹H NMR-data:

s=singlet, d=doublet, t=triplet, b=broad, J=coupling constant, δ in ppm.

### §2. Synthesis

Compounds prepared:

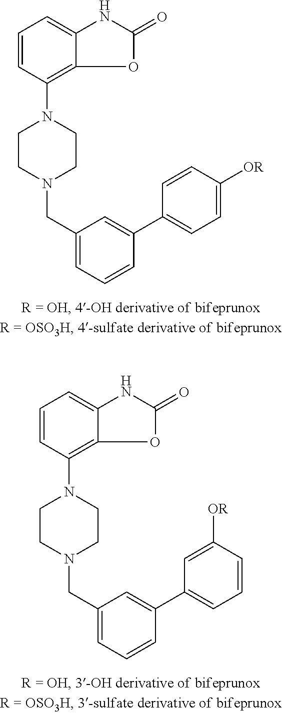

### Synthesis of the 4′-hydroxy and 4′-sulfate derivatives of bifeprunox

### Step 1

### Synthesis of 4′-hydroxy biphenyl-3-carboxaldehyde

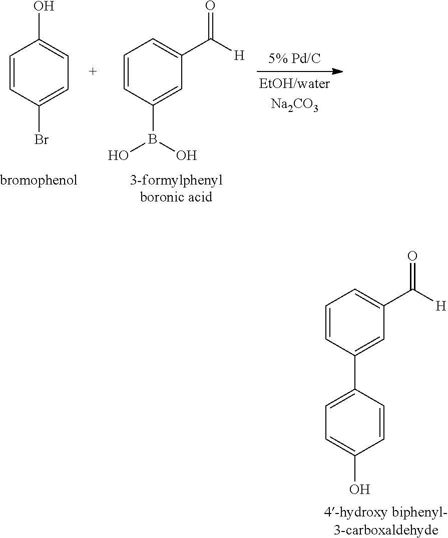

29.0 g of 4-bromophenol and 25.0 g 3-formylphenylboronic acid were dissolved in 200 ml of 96% EtOH at room temperature. Under stirring, 35.3 g of Na₂CO₃ and 100 ml of water were added. An amount of 6.0 g of Pd/C was added, rinsed with 50 ml of 96% EtOH. The mixture was heated to reflux 80° C., and kept 79-81° C. for 1 hour. Subsequently, 150 ml of water was added, resulting in a drop of the temperature to 60° C. The mixture was filtered hot over a 2 cm bead of high flow and rinsed with 200 ml of EtOAc (bright yellow filtrate). The filtrate was evaporated to dryness and 200 ml of EtOAc and 100 ml of water were added. The layers were separated and the water layer was extracted with EtOAc (twice, with 100 ml and 50 ml, respectively) (strong decolourisation). The combined organic layers were washed with 150 ml of water and then with 100 ml of brine. The solvent was evaporated from the off-white coloured organic layer to give 33.61 g (101.5% c/c) of off-white coloured solid. The solid was dissolved in 100 ml of DEM by heating, followed by cooling to room temperature in about 60 min. (crystallisation started at 70-75° C., no crust formation). Then the mixture was stirred for 1 hour at 0° C., filtered and washed twice with 25 ml of cold DEM (<−5° C.). The precipitate was dried under vacuum at 40° C. to give 21.66 g of an off-white coloured solid. Further work-up: the filtrate was evaporated to give 12.83 g off-white/yellow oil/solid. This was stirred in 30 ml of DEM at 0° C. for 1 hour. After filtration, the solid was washed twice with 5 ml of cold DEM and dried under vacuum at 40° C. to give 1.72 g of an off-white coloured solid. Total yield 21.66+1.72=23.38 g.

### Step 2

### Synthesis of 4′-hydroxy-bifeprunox

### 7-[4-(4′-hydroxy[1,1′-biphenyl]-3-ylmethyl)-1-piperazinyl]-2(3H)-benzoxazolone

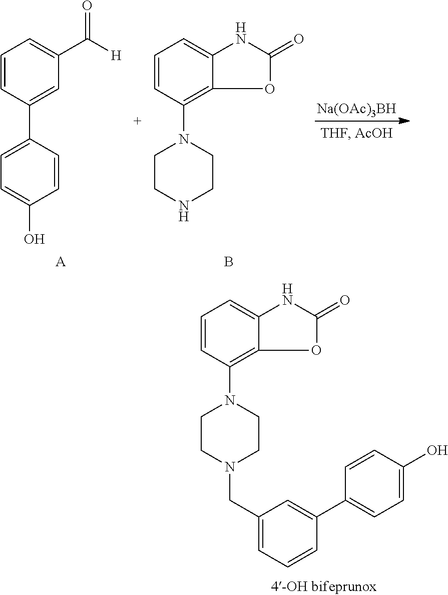

Into a reactor 200 ml of THF was charged under a nitrogen atmosphere. 45.08 g of compound B and 21.38 g of aldehyde A were added, followed by 600 ml of THF (suspension). Under stirring at room temperature 4.2 g of Na(OAc)₃BH (portion 1) and 1.0 ml of AcOH were added. After 45 minutes 4.2 g of Na(OAc)₃BH were added, which was repeated after 90, 150, 225, 270 and 360 minutes. After the last addition stirring was continued for at least 2 hours. Then, another 10.67 g of aldehyde A was added. After 45 minutes 4.2 g of Na(OAc)₃BH were added, which was repeated after 90, 150, 225 minutes. Stirring continued for at least 8 hours. The suspension was filtered over a P3 filter (φ 10 cm) and the filter cake was washed with 100 ml of THF and 100 ml of 100% EtOH. The filtrate was evaporated to a thick oil/solid. Under stirring, 350 ml of EtOAc, 200 ml of water and 50 ml of 10% of Na₂CO₃ were added, continued by stirring for 5 minutes. The layers were separated and the water layer was extracted with 100 ml of EtOAc. The combined organic layers were washed with 100 ml of water and 100 ml of 2.5% of Na₂CO₃, respectively. The organic layers were evaporated till dry to afford an off-white coloured solid. The solid was stirred in 200 ml of 100% EtOH for 30 minutes at room temperature. The solid was filtered off and the filter cake was washed with 50 ml of cold 100% EtOH. The product was dried under vacuum at 40° C. to afford 48.1 g of an off-white coloured product with melting point: 211-213° C.

¹H NMR (400 MHz, DMSO-d6/CDCl₃ 4/1):

δ=11.30 (s, 1H; NH), 9.50 (bs, 1H; OH), 7.51 (s, 1H; ArH), 7.49-7.41 (m, 3H; ArH), 7.36 (t, ¹J (H, H)=8.0 Hz, 1H; ArH), 7.24 (d, ¹J (H, H)=8.0 Hz, 1H; ArH), 6.99 (t, ¹J (H, H)=8.0 Hz, 1H; ArH), 6.85 (d, ¹J (H, H)=8.0 Hz, 2H; ArH), 6.61 (d, ¹J (H, H)=8.0 Hz, 1H; ArH), 6.57 (d, ¹J (H, H)=8.0 Hz, 1H; ArH), 3.59 (s, 2H; NCH₂Ar), 3.22 (bs, 4H; NCH₂), 2.59 (bs, 4H; NCH₂).

The mother liquor was evaporated to afford 16 g of oil/solid.

### Step 3

### Synthesis of 4′-sulfate-bifeprunox

### 7-[4-(4′-sulfooxy[1,1′-biphenyl]-3-ylmethyl)-1-piperazinyl]-2(3H)-benzoxazolone

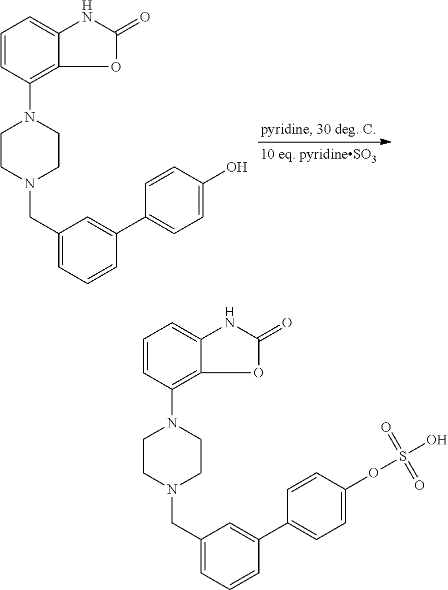

The reactor was charged with 15.9 g of pyridine.SO₃ and 30 ml pyridine. The resulting white suspension was heated on an oil bath to 30° C. (±4° C.). A solution was prepared of 4.01 g of the phenolic compound in 25 ml of pyridine (clear yellow solution). Under stirring the solution was added dropwise to the suspension in ±4 hours. The reaction mixture was stirred for 1 hour (bottom of reactor shows half liquid/solid). Then, 50 ml of DCM and 10 ml of water were added. In an ice bath the mixture was cooled and 9.24 g of NaHCO₃ were added in portions (gas evolution, T<15° C.). The resulting mixture was stirred for half an hour. Then, the reaction mixture was evaporated to dryness. The residue was stirred at room temperature for 1 hour in 100 ml of 100% EtOH. The mixture was filtered with suction and the filter cake was washed with 25 ml of EtOH. The residual solid was stirred in 50 ml of 5% NaHCO₃ for 1 hour. The reaction mixture was filtered (very slowly) and the solid was washed with 5 ml of water. The solid was under vacuum at 40° C. to afford 4.58 g of a nearly white solid.

¹H NMR (400 MHz, DMSO-d6/CDCl₃ 4/1):

δ=11.55 (s, 1H; NH), 9.70 (bs, 1H; OSO₃H), 7.82 (s, 1H; ArH), 7.72 (d, ¹J (H, H)=8.0 Hz, 1H; ArH), 7.61 (d, ¹J (H, H)=8.0 Hz, 2H; ArH), 7.54 (t, ¹J (H, H)=8.0 Hz, 1H; ArH), 7.48 (d, ¹J (H, H)=8.0 Hz, 1H; ArH), 7.34 (d, ¹J (H, H)=8.0 Hz, 2H; ArH), 7.04 (t, ¹J (H, H)=8.0 Hz, 1H; ArH), 6.70 (d, ¹J (H, H)=8.0 Hz, 1H; ArH), 6.68 (d, ¹J (H, H)=8.0 Hz, 1H; ArH), 4.48 (s, 2H; NCH₂Ar), 3.80-3.00 (bm, 8H; 2×NCH₂).

### Synthesis of the 3′-hydroxy and 3′-sulfate derivatives of bifeprunox

The 3′-hydroxy and 3′-sulfate derivatives of bifeprunox (7-[4-(3′-hydroxy[1,1′-biphenyl]-3-ylmethyl)-1-piperazinyl]-2(3H)-benzoxazolone and 7-[4-(3′-sulfooxy[1,1′-biphenyl]-3-ylmethyl)-1-piperazinyl]-2(3H)-benzoxazolone, respectively) were prepared in a similar fashion as the 4-substituted derivatives.

### NMR Data:

3′-hydroxy-bifeprunox. ¹H NMR (400 MHz, DMSO-d6/CDCl₃ 4/1):

δ=11.30 (s, 1H; NH), 9.45 (bs, 1H; OH), 7.54 (s, 1H; ArH), 7.47 (d, 1H; ArH), 7.39 (t, ¹J (H, H)=8.0 Hz, 1H; ArH), 7.31 (d, ¹J (H, H)=8.0 Hz, 1H; ArH), 7.23 (t, ¹J (H, H)=8.0 Hz, 1H; ArH), 7.08-7.00 (m, 2H; ArH), 6.98 (d, ¹J (H, H)=8.0 Hz, 1H; ArH), 6.76 (d, ¹J (H, H)=8.0 Hz, 1H; ArH), 6.61 (d, ¹J (H, H)=8.0 Hz, 1H; ArH), 6.57 (d, ¹J (H, H)=8.0 Hz, 1H; ArH), 3.61 (s, 2H; NCH₂Ar), 3.23 (bs, 4H; NCH₂), 2.62 (bs, 4H; NCH₂).

3′-sulfate-bifeprunox. ¹H NMR (400 MHz, DMSO-d6/CDCl₃ 4/1):

δ=11.58 (s, 1H; NH), 9.80 (bs, 1H; OSO₃H), 7.85 (bs, 1H; ArH), 7.72 (bd, ¹J (H, H)=8.0 Hz, 1H; ArH), 7.60-7.48 (bm, 3H; ArH), 7.42-7.36 (bm, 2H; ArH), 7.24 (bs, 1H; ArH), 7.04 (t, ¹J (H, H)=8.0 Hz, 1H; ArH), 6.69 (d, ¹J (H, H)=8.0 Hz, 1H; ArH), 6.65 (d, ¹J (H, H)=8.0 Hz, 1H; ArH), 4.47 (s, 2H; NCH₂Ar), 3.85-3.00 (bm, 8H; 2×NCH₂).

### §3. Pharmacological Tests

The potency of 3′-hydroxy-bifeprunox, 4′-hydroxy-bifeprunox, 3′-sulfate-bifeprunox, 4′-sulfate-bifeprunox and, for comparison, bifeprunox as a ligand on the human dopamine D_{2L} receptor was investigated using a receptor binding assay and determination of functional activity was investigated using a cAMP assay. All compounds were dissolved in DMSO.

### Method—Receptor Binding

The binding of the compounds of the invention to the human D_{2L} receptor was performed using displacement of radioactive-labeled [³H]-spiperone binding on membranes prepared from Chinese hamster ovary (CHO) cells expressing the recombinant human D_{2L} receptor.

Human dopamine D_{2L} receptors were cloned in CHO-K1 cells and obtained from Dr. D. Grandy, Vollum Institute, Portland, Oreg., USA.

The binding assay has been carried out using [³H]-spiperone as ligand (see Eur. J. Pharmacol. 46:377-381, 1977). The labeled compound was incubated with or without the test compound, with a preparation from cloned CHO cells carrying the human dopamine D_{2L} receptor. Separation of bound and free ligand was performed by filtration over glassfiber-filters. After two washings, the bound-fraction remained on the filter. Radioactivity trapped on the filter was measured by scintillation counting. Results are expressed as pKᵢ (negative logarithm of the inhibition constant Kᵢ which is calculated form the IC₅₀). See Table 1.

### Method—Measurement of Adenylate Cyclase Activity

Cells were grown in a α-DMEM culture medium, supplemented with 10% heat-inactivated foetal calf serum, 2 mM glutamine, 1 mM pyruvate, 200 μM G418 at 37° C. in 93% air/7% CO₂. For incubation with test compounds (concentrations 10⁻⁶-10⁻¹⁰M), confluent cultures grown in 24 wells plates were used. Each condition or substance was routinely tested in quadruplicate. Cells were loaded with 1 μCi [³H]-adenine in 0.5 ml medium/well. After 2 hours, cultures were washed with 0.5 ml PBS containing 1 nM IBMX and forskolin with or without test compound. After aspiration the reaction was stopped with 1 ml trichloroacetic acid 5% (w/v). The [³H]ATP and [³H]cAMP formed in the cellular extract were assayed as described by Salomom et al. (Anal. Biochemistry 58: 541-548, 1974) and Weiss et al. (J. Neurochem. 45: 869-874, 1985). The extract was passed over Dowex (50W-4 200-400 mesh) and aluminiumoxide columns, eluted with water and 0.1M imidazole (pH=7.5). Eluates were mixed with 7 ml of Insta-gel and radioactivity was counted with a scintillation counter. The conversion of the cAMP fraction as compared to combined radioactivity in both cAMP and ATP fractions, and basal activity was subtracted to correct for spontaneous activity.

Dopamine D_{2L}: reference compound: quinpirole; incubation time: 20 min.

### Data Analysis

Subsequently, the mean of four observations was taken as an estimate for drug-induced, receptor-mediated effects at specified messenger accumulation, expressed as percentage of control values (forskolin-stimulated cAMP accumulation).

By using the non-linear curve-fitting program XL-FIT mean values were plotted against drug concentration (in molar) and sigmoid curve (four parameter logistic curve) was constructed. The maximal forskolin-induced stimulated conversion was taken as maximum value and the maximal inhibition as minimum and these values were fixed during the fitting process. The concentrations of the test compound, causing 50% of the maximally obtained inhibition of forskolin-induced cAMP accumulation (EC₅₀), are averaged over several experiments and presented in Table 2 as mean pEC₅₀±s.e.m. As a full dopamine D_{2L} agonist, quinpirole was used.

TABLE 1
Receptor binding data of bifeprunox and 4 derivatives thereof

| | pKᵢ value | Number of |
|---|---|---|
| Test compound | mean ± s.e.m. | experiments |
| Bifeprunox | 7.4 (0.2) | 7 |
| 4′-OH | 7.9 (0.4) | 4 |
| 3′-OH | 7.9 (0.2) | 4 |
| 4′-sulfate | 8.2 (0.1) | 4 |
| 3′-sulfate | 7.9 (0.3) | 4 |

Conclusion: compared to bifeprunox, the 4 derivates exhibit higher affinities to the dopamine D_{2L} receptor.

TABLE 2
Dopamine D_{2L} agonistic effects

| Test | Conversion of cAMP, % of control (mean (±s.e.m.)) | | | | | | pEC₅₀ mean | PS |
|---|---|---|---|---|---|---|---|---|
| compound | 10⁻¹⁰M | 10⁻⁹M | 10⁻⁸M | 10⁻⁷M | 10⁻⁶M | 10⁻⁵M | (±s.e.m.) | (%) |
| Quinpirole | — | 100 (4) | 91 (6) | 51 (5) | 16 (4) | 0 (0) | 7.0 (0.1) | 84 (a) |
| | | n = 9 | n = 8 | n = 9 | n = 9 | n = 9 | n = 9 | |
| Bifeprunox | 95 (15) | 94 (13) | 84 (12) | 69 (11) | 69 (10) | — | 8.2 (0.3) | 31 |
| | n = 5 | n = 6 | n = 6 | n = 5 | n = 6 | | n = 6 | |
| 4′-OH | 90 (9) | 59 (3) | 58 (4) | 50 (2) | 45 (4) | — | 9.7 (0.2) | 55 |
| | n = 3 | n = 3 | n = 3 | n = 3 | n = 3 | | n = 3 | |
| 3′-OH | 84 (4) | 70 (6) | 63 (7) | 56 (8) | 51 (10) | — | 9.1 (0.5) | 49 |
| | n = 4 | n = 4 | n = 4 | n = 4 | n = 4 | | n = 3 | |
| 4′-sulfate | 85 (9) | 72 (13) | 65 (13) | 63 (14) | 51 (16) | — | 9.2 | 49 |
| | n = 3 | n = 3 | n = 3 | n = 3 | n = 3 | | n = 2 | |
| 3′-sulfate | 94 (5) | 93 (9) | 79 (6) | 66 (12) | 49 (6) | — | 7.9 (0.5) | 51 |
| | n = 3 | n = 3 | n = 3 | n = 3 | n = 3 | | n = 3 | |

(a) 10 μM: 100%
n = number of experiments
PS: mean % stimulation (agonism) at 1 μM

Conclusion: the 4 derivates exhibit a significantly higher level of dopamine D_{2L} agonistic activity at 1 μM than bifeprunox (49-55% and 31%, respectively).

1-12. (canceled)

13. A compound of formula (I) 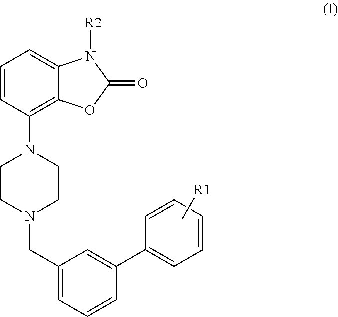
or an N-oxide or a pharmaceutically acceptable salt, or a solvate or hydrate of any of the foregoing,
wherein
R1 is chosen from 3-OH, 4-OH, 3-OSO₃H and 4-OSO₃H; and
R2 is H.

14. The compound of claim 13, wherein R1 is 3-OH or 4-OH and R2 is H.

15. The compound of claim 13, wherein R1 is 3-OSO₃H or 4-OSO₃H, and R2 is H.

16. A process for preparing a compound of formula 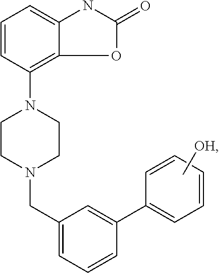
the process comprising reacting a compound of formula 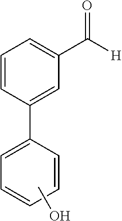
with 7-piperazin-1-yl-3H-benzooxazol-2-one hydrochloride.

17. A process for preparing a compound of formula 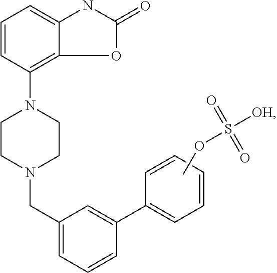
the process comprising reacting a compound of formula 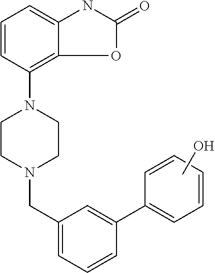
with an excess of pyridine SO₃.

18. A pharmaceutical composition comprising a compound of formula (I) 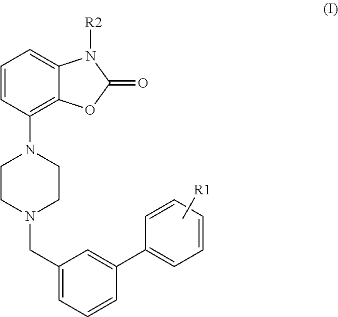
or an N-oxide or a pharmaceutically acceptable salt, or a solvate or hydrate of any of the foregoing,
wherein
R1 is chosen from 3-OH, 4-OH, 3-OSO₃H and 4-OSO₃H; and
R2 is H; and
a pharmaceutically acceptable auxiliary.

19. A method for treating or alleviating a D₂ receptor mediated disease or condition, wherein D₂ receptor agonistic effects are needed, the method comprising administering a compound of formula (I) 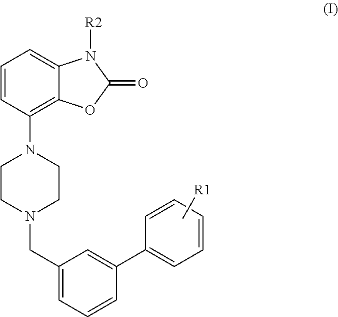
or an N-oxide or a pharmaceutically acceptable salt, or a solvate or hydrate of any of the foregoing,
wherein
R1 is chosen from 3-OH, 4-OH, 3-OSO₃H and 4-OSO₃H; and
R2 is H,
to a patient in need of said treatment or alleviation.

20. The method of claim 19, wherein the D₂ receptor mediated disease or condition is a CNS-related disease or condition.

21. The method of claim 20, wherein the CNS-related disease or condition is chosen from Parkinson's disease and Restless Leg Syndrome.

22. The method of claim 19, wherein the D₂ receptor mediated disease or condition is a non-CNS disease or condition.

23. The method of claim 22, wherein the non-CNS disease or condition is chosen from hypertension, acromegaly resulting from the hypersecretion of growth hormone caused by pituitary adenomas, hyperprolactinaemia, hyperprolactinaemia caused by administration of typical neuroleptic drugs, atypical antipsychotics and other dopamine D₂ receptor antagonists, ovarian hyperstimulation syndrome, cell proliferation in small-cell lung carcinomas, and multi-drug resistance in cancer chemotherapy and in dermatology.
